(19) United States
(12) Patent Application Publication
(10) Pub. No.: US 2026/0364284 A1
(43) Pub. Date: Nov. 19, 2026

(54) NASAL AND PULMONARY DRUG DELIVERY

(71) Applicant: ENVIROHALE MALTA HOLDINGS LIMITED, Pieta, PTA (MT)

(72) Inventors: William Samuel Hunter, Victoria VIC (AU); David Alexander Vodden Morton, Victoria VIC (AU)

(21) Appl. No.: 19/515,449

(22) PCT Filed: Aug. 29, 2024
(86) PCT No.: PCT/IB2024/058393

§ 371 Date: Feb. 27, 2026

(30) Foreign Application Priority Data
Aug. 30, 2023 (GB) 2313218.6

(51) Int. Cl. A61M 15/00; A61M 11/02; A61M 15/08

(52) U.S. Cl. CPC A61M 15/009; A61M 11/02; A61M 15/0035; A61M 15/0051; A61M 15/08; A61M 2202/064

(57) ABSTRACT

A device for delivering a drug to a user via a nasal or pulmonary route is disclosed. The device includes a propellant energy source to deliver the drug. The propellant energy source is or comprises an aerial gas that is stored in a canister separate of the drug, at a pressure of at least 2 Bar, adsorbed on an adsorbant where it is released therefrom to dispense the drug. The device is absent of a bag-on-valve in the canister.

This invention relates to nasal and pulmonary drug delivery. More particularly it relates to novel drug delivery devices and methods of delivering drugs via the nasal and oral pulmonary routes. It also relates to a power source comprising a canister of up to 50 ml, more preferably up to 35 ml and typically from 10 to 22 ml with a gas volume metering valve designed to deliver from 0.1 ml to 5 ml, more preferably 0.2 to 2.5 ml of a propellant gas at a pressure of from 2-10 Bar. The term “drug” includes both prescription pharmaceuticals (including biologics) and other functional actives delived to a user and includes both the pure form, salts and formulations thereof. The term “approved drug”, in contrast, is limited to a regulatory approved drug (including a biologic), namely one approved by e.g. the United States Food and Drug Administration (FDA), European Medicines Agency (EMA), or another national government agency.

### BACKGROUND

Novel Platforms for drug delivery Applications, Woodhead Publishing Series in Biomaterials, 2023, pages 568-606,

https://www.sciencedirect.com/science/article/pii/B9780323913768000197 teaches nasal and pulmonary drug delivery are attractive routes for the administration of a growing number of drugs for topical and systemic treatment as well as for prevention by vaccines. This systemic treatment is of particular interest for drugs with poor bioavailability, as the gastrointestinal passage and hepatic first pass effect can be avoided.

As outlined in a recent review article, Frontiers in Drug Delivery, 13 Apr. 2022, Respiratory Drug Delivery, Vol 2, 2022, Frontiers|Half a Century of Technological Advances in Pulmonary Drug Delivery: A Personal Perspective (frontiersin. org) inhalation therapy has existed for over 2000 years.

The basic pulmonary delivery technologies that have evolved over the past half century can best be described as small personal portable aerosol generators. Indeed, the pulmonary respiratory market was worth $53b in 2021 (DataBridge Market Research).

They can be divided into four major classes:
Pressurised Metered Dose Inhalers (pMDI's);
Powder Dose Inhalers (PDI's);
Soft Mist Inhalers (SMI's); and
C Nebulisers.

They deliver drugs as:
Powders;
Suspensions, dispersions or emulsions; and
Solutions

These technologies have:
Different operating characteristics;
Patient interface/user requirements; and
Dose limitations.

These, devices, drug forms and technologies are illustrated in FIG. 1 herein (taken from the review article).

Devices (empty) and combination products (a device containing a drug) according to the invention differ from these traditional nasal and pulmonary inhalers on a number of levels, and FIGS. 2 to 6 illustrate some prior art devices to assist in highlighting key differences. These prior art devices are however briefly identified below:

### Pressurised Metered Dose Inhalers (pMDIs)

A simple pressurised metered dose inhaler of the art, is illustrated in FIG. 2, and comprises as the propellant power source, a liquid (and a gas headspace) which is mixed with the drug in the form of a suspension or solution. A metered dose is delivered on actuation and the device can include a mouthpiece or a nasal adapter. The aerosol is created by expansion of the liquid propellant.

Recognised advantages of pMDI-based inhalation systems are their portable nature, and single step ease of operation.

A major patient-related use issue with these devices is the requirement for a coordinated inhalation and actuation manoeuvre. The pMDI is an active device, delivering a single dose in a fast plume (about 100 m/s at point of generation) over a period of microseconds. The pMDI creates the fast moving plume from the expansion of the evaporating propellant at the actuator orifice. The velocity and timing are known challenges in patient coordination of inhalation with firing the device. For pMDIs, the patient needs to coordinate the actuation of the fast spray with their steady slow inhalation. If the patient inhales before or after actuation, the majority of the medicament is likely to impact in the throat and be swallowed as a recent review found in the case of pMDIs, 87% of patients demonstrated such poor technique—J Allergy Clin Immunol Pract. 2022 Jul; 10(7):1813-1824.

To overcome such issues, two approaches have been taken: spacers and breath-actuated pMDIs. Both attempts to overcome these issues present their own complexities and disadvantages.

pMDI devices have a smaller delivery volume than their DPI and nebulizer counterparts (limiting the range of therapeutic doses). Patients are reported to dislike their cooling “Freon” effect, due to the rapid evaporation of the propellant. With changes in propellant from CFC to HFA, and with new different HFAs-patients reported experience of a change in taste and sensation, and associated this with inhaler not working, so reduced use.

In addition, in use the pMDI suffers from a potential loss of prime, of the liquid-based dose from the metering valve. This metering valve relies on consistent gravimetric liquid fill. If the device is not appropriately shaken and held upright, liquid dose filling of the valve can be reduced with a resulting failure to deliver the correct dose. For suspension formulations, users must shake the canister sufficiently and reproducibly to also ensure that the suspension is resuspended to a homogenous state, and ensure the required dose is delivered.

HFA propellants, existing as liquids in the compressed state can act as solvents within the cannister, and a significant concern with all such materials is the potential to extract and leach chemicals from polymeric valve materials. The propellants are in contact with all internal canister and valve components, and are also in contact with the drug substances and excipients, all of which which must be proven to not cause adhesion, create contaminants and degradation products and cause swelling of gaskets.

All these issues are either not or are less relevant for the current invention where the drug is metered external to the cannister, and the propellant is a gas and not a liquid.

A major driver of technology in this sector has been legislation. In the mid-1990s, chlorofluorocarbon (CFC) propellants were banned due to their ozone depletion potential (Federal Register, 1994). The realization that pMDIs, then as now the mainstay of inhalation therapy, might no longer be available, sparked enormous efforts to find alternatives, as exemplified in patent filings of that period. Between 1990 and 2010, nearly 1,000 patents describing inhalation technologies were filed, compared to less than 100 in the previous two decades (Stein and Theil, J Aerosol Med Pulm Drug Delivery 2017 Feb;30(1):20-41.doi: 10.1089/jamp.2016.1297).

However, this drive for new technologies was short lived as alternative propellants were developed through significant efforts and investments and the pMDI remains the mainstay of inhalation therapy to this day.

However, the replacement for CFCs, Hydro Fluorocarbon Alkanes (HFAs) continue to be a major contributer to global warming having up to 3350 times the global warming potential (hereon GWP) of carbon dioxide (CO₂). (On Drug Delivery, April 24, 2023, Issue 145, page 13). It is known that a single pMDI can release the equivalent of 25 kg of CO₂ from one canister.

Indeed, England's National Health Service (NHS) has identifed metered-dose inhalers to be responsible for 3 percent of the total emissions and 13 percent of primary care emissions by the NHS and Pharmaceutical manufacturer GSK has estimated that metered-dose inhalers were responsible for 45 percent of their carbon emissions.—see e.g. The Climate is Changing for Metered-Dose Inhalers and Action is Needed, J. Pritchard, Drug Design, Development and Therapy 2020:14 3043-3055.

Accordingly there remains a clear and urgent need for pMDI technologies that are more sustainable.

In light of the global warming problems associated with HFA propellants, the industry has developed alternative HFA propellants. For example HFA152a is an alternative HFA propellant which has 138 times the GWP of CO₂. However the problem with such alternate propellants is that they will take extensive time and cost to re-validate. Such re-validation needs to be performed with each one of the wide range of pharmaceutical compounds they need to be re-formulated with. Hence there are very large challenges with this overall re-formulation approach in terms of toxicology studies, stability, extractables and leachables, propellant flammability, and how they cause changes in drug deposition to the lungs. (On Drug Delivery, April 24 2923, Issue 145, pages 13-17).

Multiple issues cloud both current and the two new HFA propellants proposed namely HFA-152a and HF0-1234ze(E), as a category they are reported with pharmacological and toxic effects—Sellers, Allergy Asthma Clin Immunol (2017) 13:30, DOI 10.1186/s13223-017-0202-0. Current pressurised metered dose asthma inhaler (pMDI) propellants (HFAs) are not inert pharmacologically, with the compound family having smooth muscle relaxant effect and anaesthetic effects. New propellant HFA152a has been associated with deaths through abuse caused by deliberate inhalation of this gas from consumer aerosols—The Climate is Changing for Metered-Dose Inhalers and Action is Needed, J. Pritchard, Drug Design, Development and Therapy 2020:14 3043-3055.

The new HFAs are very different in physicochemical properties, more combustible, and chemically very stable. Thus, they are polyfluoroalkyl substances (PFAS), “forever” chemicals, which are also associated with significant environmental impact issues, and if degraded by heat produce dangerous products such as hydrogen fluoride (HF).

The Organization for Economic Cooperation and Development (OECD) define PFAS to include medical grade fluorinated gases that can be used as propellants. Germany, the Netherlands, Denmark, Norway and Sweden have proposed restrictions on PFAS in Europe, which will likely involve a more stringent timeline in regulations banning their use—A. Lee, “Developing the Next Generation of Inhalers” Technology Networks July 2023.

pMDIs have been cheaper to produce than current DPIs, so there is concern over affordability to switch patients into DPIs. However, the cost of medical HFA propellants will rise by at least 6-fold as their regulation drives down uses—so they will become more expensive (J. Pritchard, Drug Design, Development and Therapy 2020:14 3043-3055; Wilkinson and Woodcock, Br J Clin Pharmacol. 2022; 88:3016-3022.)

In contrast Applicant has a unique power source, containing an aerial gas adsorbed on an adsorbent in a canister. The aerial propellant gas can aerosolise a unit dose of a powder or a liquid which are contained separate of the power source. The aerial propellant gas has zero GWP. Because the aerial propellant is simply used to entrain the drug in an aerial gas stream, there are none of the prior art problems associated with re-formulation to alternative HFA propellants.

### Dry Powder Inhalers (DPIs)

Dry powder inhalers take multiple forms.

Passive devices (all current commercial DPI devices) rely on a patients inhalation efforts to entrain, disperse and deliver the powder to the airways.

Active devices employ mechanical or electrical technology, in addition to patient inhalation to entrain, disperse and deliver the powder to the airways.

Devices are further split into “carrier” based systems, which can deliver a single dose or multiple doses from a reservoir, and “agglomerate” based systems.

Single dose devices include capsule based formulations such as Aerolizer® (Novartis) and Handihaler® (Boehringer Ingelheim) and Multi-dose devices. These multi-dose devices include devices with multiple unit doses, such as, the Diskhaler® and Diskus® (Glaxo Smith Kline) and reservoir devices, such as, the Turbohaler (Astra Zeneca) and the Pulvinal® (Cheisi).

Obviously the designs vary with type, but common essential features are:
An inlet through which air is drawn;
A metered dosing chamber, where the powdered drug is stored prior to actuation; and
An outlet from which the dose is delivered to the user.

WO2024033662 discusses some of the challenges associated with current dry powder inhalers. Passive devices (all current devices) rely on harnessing a proportion of the available energy from the patient's inhalation to do work on the powdered formulation, to break up and resuspend the powdered medicament, to further deagglomerate the particles and produce a fine, respirable aerosol.

Many marketed leading DPIs only achieve 20 to 30% Fine Particle Fraction (FPF) of below 5 μm aerodynamic diameter. The remaining drug is agglomerated or attached to the larger carrier particles. Indeed, WO2024033662 states:
“A huge challenge for all current DPIs is that because they are solely reliant upon harnessing energy from the patient's inspiratory manoeuvre, it is very difficult to achieve consistent delivery of drug when the energy available varies considerably from patient to patient.”

Whilst “active” DPIs are, and have been, in development, right now there are none commercially available. The appeal of an active DPI is to overcome the huge variability from one user to another by containing an internal energy source to produce a respirable aerosol, which is independent of the user, or more specifically, it is independent of how the user inhales.

Indeed, WO2024033662 states:
“There are probably no active DPIs available because they are so complicated to design, optimise and produce. The ideal DPI system comprising a deagglomeration engine, or deagglomeration apparatus, is a system that consistently produces a high fine particle fraction, independently of how the user inhales, and is simple and cost effective to manufacture.”

Applicants use of their power source addresses these challenges and obviates the need for complexity and cost which limited successful commercialization of devices such as disclosed in U.S. Pat. No. 6,257,233 by Nektar.

One such prior art design is illustrated in FIG. 3 which illustrates a multi-dose passive device. A significant problem with such prior passive dry powder inhalers, as outlined above, is that the user's inspiratory air flow varies widely across the user base. This results in a substantial lack of dosing repeatability. Furthermore, for elderly patients and paediatric patients, such users typically cannot generate sufficient inspiratory effort and coordination to trigger entrainment and deagglomeration of powder to provide the intended aerosolised dose quantity and form. Indeed, clinical practice guidelines recognise this such that for certain patient groups only pMDI's are prescribed.

For an active device there is also provided:
An auxillary energy source.

The majority of active devices rely on pneumatic forces arising from manually operated pumps that are primed immediately prior to inhalation with compressed air and released when the patients flow stream has been sensed by a detector incorporated into the device. Alternatively, the pneumatic source may also be actuated simultaneously with inhalation by the user.

Once such prior art design is illustrated in FIG. 4. The design shown is a single-dose device. This design uses a manually-powered piston pump to provide an entrainment air stream. An issue with such devices is that patients may not have sufficient dexterity or strength to prime the pump, as significant force is required to presurise a small volume of air. The pump mechanism is also complex, in order to minimise the effort required, adding significant physical size and a high cost to the device manufacture.

In contrast Applicant has as a power source, a gas adsorbed on an adsorbent in a canister. This canister provides a substantially constant pressure source and flow rate of entrainment gas over a defined working life of the canister. As such it can be used to provide a simple active DPI without the complexity and repeatability problems of the prior art. The device size and complexity can be minimised and the drug can be delivered to the lungs with minimal operation and inspiratory effort by the user.

The gas is stored in a canister, at a pressure of at least 2 Bar, adsorbed on an adsorbant where it is released therefrom to dispense the drug. A filter or frit ensures the adsorbant is retained in the canister and does not exit with the gas.

### Soft Mist Inhaler (SMIs)

A prior art soft mist inhaler, as illustrated in FIG. 5, aerosolises a drug solution by pushing the drug through a “uniblock”, which comprises an extremely small orifice. A compressed spring provides the force. The dose is delivered over a longer period, typically 1.2 sec, as compared to 0.5 sec with a pMDI. One of the main problems with such devices is that they are only able to deliver a small quantity of liquids, e.g. typically 15 μl (compared to e.g. 25 to 100 μl with a pMDI and over 10 mg with a DPI). In many cases it is desired to deliver much higher masses of drugs than can be solubilised in such a small liquid volume.

In contrast Applicant has as a power source, a gas adsorbed on an adsorbent in a canister. This enables drugs to be delivered potentially in powder form from a capsule, which may contain at least an order of magnitude more drug (for example, up to 50 mg of powder) than is possible with an SMI.

Each of the three device types described above may be used in combination with a Valved Holding Chamber (VHC) or Spacer device which hold the aerosol until inhalation.

### Nebuliser

A prior art nebuliser, as illustrated in FIG. 6, uses a compressor to continuously aerosolise a liquid solution or suspension containing a drug, present in a reservoir, to the user. Hand held nebulisers are also used, which can use a vibrating mesh to aerosolise the liquid. Such devices are typically battery or electrically powered, and require expensive control electronics. Nebulisers do not deliver the drug in a single breath, but require several minutes to deliver the required dose with significant patient breath coordination and compliance challenges.

In contrast Applicant has as a non-electrical and portable power source, with a gas adsorbed on an adsorbent in a canister which can be used, e.g. pulsed to achieve the desired aerosolization.

Prior art identified, but not considered relevant includes US2014/0048566 and US 2006/0049215 both of which incorporate a secondary container with activated carbon into a cannister filed with a liquid to automatically repressurise the head space above the liquid.

### BRIEF SUMMARY OF THE DISCLOSURE

In accordance with a first aspect of the invention there is provided a device for delivering a drug to a user via the nasal or pulmonary route wherein the device comprises a propellant energy source to deliver the drug, which propellant energy source is or comprises an aerial gas that is stored in a canister separate of the drug, at a pressure of at least 2 Bar, adsorbed on an adsorbent where it is released therefrom to dispense the drug, which device is absent of a bag-on-valve.

In one embodiment the propellant energy source which is separate of the drug is positioned in the device upstream of the drug, such that on actuation of the canister a unit dose of the drug is aerosolised from the device in a controlled manner.

Bag on valve technology, and modifications thereof, are disclosed in WO2020021473 which document is incorporated by reference.

Preferably the pressure is between 2 and 16 Bar at 25 °C, more preferably still between 2 and 10 Bar and most preferably between 4 and 8 Bar.

Preferably the adsorbent is an activated carbon or a functionalised activated carbon.

The activated carbon may be prepared from one of a host of carbon sources including, among others, natural carbonaceous sources, such as peat, wood, coal, nutshell (such as coconut), petroleum coke, bone, and bamboo shoot, drupe stones and various seeds; and synthetic sources, such as poly(acrylonitrile) or phenol-formaldehyde. The carbon is activated to develop an intricate network of pores and surface area sufficient for adsorption. The pores have various sizes ranging from microporous to sub-microporous dimensions of molecular-sized entities. The larger transport pores provide access to the smaller pores in which most of the adsorption of propellant, such as gaseous species, takes place. Carbon activation is conducted with gaseous activation using steam, carbon dioxide or other gases at elevated temperatures, or chemical activation using, for example, zinc chloride or phosphoric acid. Other activation processes may be used to achieve the pore structure and surface area that provides an extensive physical adsorption property and a high volume of adsorbing porosity.

For embodiments of the invention, the activated carbon is prepared to contain a relatively high prevalence of micropores and a low enthalpy of adsorption. This is to enable a substantially maximum gas delivery. The size of the micropores ranges from about 0.5 nm to about 2.5 nm. In an embodiment, the micropores are about 1.0 to 2.0 nm. The enthalpy of adsorption is less than about 25 kJ (per mole of adsorbate). In other words, a carbon with a high capacity uptake for the compressed gas and a low retention (or heel) on discharge provides for the maximum gas volume delivery. For a high uptake, the activated carbon has a high concentration of micropores. For a low retention, carbons with a low enthalpy of adsorption (for the particular gas) are selected as there is a relatively good correlation between these two variables. Unlike traditional dispensing systems that rely on adsorbed permanent gases, application of activated carbon in embodiments of the present invention enables propellant/gases to condense or immobilize resulting in increased gas storage and delivery capacity. Ordinarily, gas storage is accomplished by increasing the pressure in a fixed volume container and the amount of gas in the container, under non-extreme conditions, basically follows the ideal gas laws. Embodiments of the present container can physically deliver more gas than a non-carbon-filled container despite the volume lost to the carbon skeleton.

The activated carbon can be in a variety of forms, most commonly as powdered, granular or pelleted products.

Preferably the aerial gas is air, oxygen, nitrogen, or carbon dioxide.

More preferably the aerial gas is carbon dioxide or air or oxygen enriched with carbon dioxide. The carbon dioxide provides a greater volume of gas in the container and thus a greater energy.

Preferably the device is, as recognised in the pharmaceutical industry, a combination product comprising the device and a regulatory approved drug.

A combination product is defined by the FDA in 21 CFR 3.2 (e) as:
a product comprised of two or more regulated components, including i.e. drug/device, biologic/device or drug/device/biologic, that are physically, chemically or otherwise combined or mixed and produced as a single entity;
two or more separate products packaged together in a single package or as a unit and comprised of drug and device products, device and biological products or biological and drug products;
a drug, device or biological product packaged separately that according to its investigational plan or proposed labelling is intended for use only with an approved individual specified drug, device or biological product where both are required to achieve the intended use, indication or effect and where upon approval of the proposed product the labelling of the approved product would need to be changed, e. g to reflect a change in intended use, dosage form, strength, route of administration, or significant change in dose; or
any investigational drug, device, or biological product packaged separately that according to its proposed labelling is for use only with another individually specified drug, device or biological product where both are required to achieve the intended use, indication or effect.

The above includes both prefilled drug/delivery systems and prefilled biologic delivery/device systems including metered dose inhalers, dry powder inhalers and nasal sprays.

Preferably the device comprises a gas delivery mechanism for releasing a sufficient volume of the gas propellant at a speed that delivers/aerosolizes a unit dose of drug.

One such gas delivery mechanism comprises a metering valve, gas metering chamber, a valve stem and an actuator.

The metering chamber preferably has a volume capacity of at least 0.1 ml, and preferably from 0.1 to 5 ml, which volume is an order of magnitude larger than that which meters a liquid propellant from a canister of up to 50 ml, more particularly 10 to 22 ml.

The gas delivery mechanism further comprises a gas entry orifice and a gas exit orifice which facilitate the filling and emptying of the gas metering chamber.

Preferably the device also comprises a drug dosing mechanism for releasing the unit dose of drug from a chamber from which it is delivered.

One such drug dosing mechanism comprises a chamber holding a unit dose of a drug upstream of the gas propellant and, for example, a piercing mechanism for releasing, e.g. a pre-metered unit dose of powder from e.g. a capsule or blister pack.

Alternatively, the drug mechanism delivers a unit dose of a powder or liquid from a bulk chamber which is mechanically extracted as a metered dose from a bulk chamber or reservoir for onward delivery by the gas propellant.

The drug is either:
a) a dry powder;
b) a multi-phase dispersion of a solid in a liquid or a liquid in a liquid; or
c) a single-phase solution.

In a first embodiment the device is a pressurised metered dose inhaler powered by the propellant energy source.

The inhaler may comprise either a mouthpiece adapter or nasal adapter depending on whether it is intended to deliver the drug via the pulmonary or nasal route.

In a second embodiment the drug is
a) a dry powder
   and the device is an active dry powder inhaler powered by the propellant energy source.

In both the first and second embodiments the drug may be provided in a bulk chamber or in a sealed unit dosage form, such as a blister pack or capsules, in which case the device comprises a drug releasing mechanism for releasing the drug, such as a piercing mechanism in the case of capsules or a blister pack or a carousel or other mechanism for transferring a unit dose from a bulk chamber to a unit dosing chamber.

The devices of the invention can be utilised to deliver a wide range of drugs due to the benefits provided by the power source and the fact they are not limited by the liquid nature of the propellant- or passive nature of metered dose inhalers contrast FIG. 1.

According to a particularly preferred embodiment, the drug is one delivered to treat a respiratory disease.

The drug agent may be delivered for local/topical or for systemic treatment.

It is particularly favoured for treating respiratory diseases. The respiratory disease may be selected from a group consisting of asthma, chronic obstructive pulmonary disease (COPD), Pulmonary Arterial Hypertension (PAH), or lung cancer. (Inhaled drug delivery for the targeted treatment of asthma, Advanced Drug Delivery Reviews 198 (2023) 114858 and Pharmaceutical Technology, 26 Aug. 2024 (MSD snags European approval for pulmonary arterial hypertension therapy.)

According to some embodiments, the dry powder agent may include a pharmaceutical that is delivered to the subject to treat an infection in the subject's lungs including bacterial infection, viral infection, such as SARS-COV-2 (COVID-19), influenza, RSV, and fungal infection.

Known anti-infectives suitable for delivery include antibiotics, such as tobramycin, antivirals, such as remdesivir, and antifungals, such as voriconazole.

According to some embodiments, the agent may include a pharmaceutical that is delivered directly into the throat and/or oesophagus of the subject.

According to some embodiments, the agent may include a pharmaceutical that is delivered directly into the nasal cavity of the subject.

Delivery to the nasal cavity can be to any nasal region, including the sinuses, but more particularly to one of two primary target regions:

The first target region is the lower region of the nasal airways, encompassing the anterior portion of the inferior and middle turbinates. This region, like all the nasal mucosal regions posterior to the nasal vestibule, is highly vascularised. It is a commonly used region for the delivery of topical and systemic drugs (Kublik, H., & Vidgren, M. T. (1998). Nasal delivery systems and their effect on deposition and absorption. Advanced Drug Delivery Reviews, 29, 157-177). The latter are quickly absorbed into the blood stream from here. Systemic and topical therapeutics include antihistamines and corticosteroids for chronic rhinosinusitis (CRS) (Ehrick, J. D., Shah, S. A., Shaw, C., Kulkarni, V. S., Coowanitwong, I., De, S., & Suman, J. D. (2013). Considerations for the development of nasal dosage forms. Sterile Product Development: Formulation, Process, Quality and Regulatory Considerations, (pp. 99-144)). This region of the nasal airways is commonly targeted by aqueous nasal spray pumps.

A second target region is the olfactory region. Previous studies have noted that nasal drug delivery potentially allows the blood-brain barrier (BBB) to be bypassed, thereby allowing drugs to be delivered directly to the central nervous system (Maaz, A., & De Bank, P. A. (2021). In Vitro Evaluation of Nasal Aerosol Depositions: An Insight for Direct Nose to Brain Drug Delivery. Pharmaceutics, 13, 1079). Two cranial nerves pass through the nasal cavity-the trigeminal and olfactory nerve. The latter is primarily of interest for intranasal drug development targeting nose-to-brain drug delivery. Consequently, the olfactory epithelium, a sub-region of the upper posterior portion of the nasal airway, has been a target for intranasal therapeutics seeking to bypass the BBB (Rigaut, C., Deruyver, L., Goole, J., Haut, B., & Lambert, P. (2022). Instillation of a dry powder in nasal casts: Parameters influencing the olfactory deposition with uni-and bi-directional devices. Frontiers in Medical Technology, 4).

According to some embodiments, the drug or a combination of drugs may be selected from a group consisting:
i. long acting muscarinic antagonists (LAMA),
ii. long acting beta agonists (LABA),
iii. short acting beta-2 agonists (SABA), and
iv. corticosteroids.

Examples include:
i) fluticasone, budesonide, mometasone, ciclesonide, beclomethasone or other corticosteroids, umeclidinium bromide, tiotopium, ipratropium, and glycorpyrronium as long-acting muscarinic antagonists (LAMA)
ii) albuterol, salbutamol or other short acting beta agonists (SABA),
iii) salmeterol, formoterol, indacaterol, vilanterol or other long acting beta agonists (LABA);
iv) mucolytics, and
v) phosphodiesterase-4 (PDE-4) inhibitors such as tanimilast, cilomilast, roflumilast, tetomilast, oglemilast, apremilast, and piclamilast.

Other drugs of particular interest for delivery using the devices of the invention include vaccines-both protein as well as RNA based vaccines.

The drugs will be formulated based on the target.

For formulations to reach the deep lung or the blood stream via inhalation, the active agent in the formulation must be in the form of very fine particles, for example, having a mass median aerodynamic diameter (MMAD) of less than 10 μm. It is well established that particles having an MMAD of greater than 10 μm are likely to impact on the walls of the throat and generally do not reach the lung. Particles having an MMAD in the region of 5 to 2 μm will generally be deposited in the respiratory bronchioles whereas particles having an MMAD in the range of 3 to 0.05 μm are likely to be deposited in the alveoli and to be absorbed into the bloodstream.

Preferably, for delivery to the lower respiratory tract or deep lung, the MMAD of the active particles is not more than 10 μm, and preferably not more than 5 μm, more preferably not more than 3 μm, and may be less than 2 μm, less than 1.5 μm or less than 1 μm. Especially for deep lung or systemic delivery, the active particles may have a size of 0.1 to 3 μm or 0.1 to 2 μm.

Ideally, at least 90% by weight of the active particles in a dry powder formulation should have an aerodynamic diameter of not more than 10 μm, preferably not more than 5 μm, more preferably not more than 3 μm, not more than 2.5 μm, not more than 2.0 μm, not more than 1.5 μm, or even not more than 1.0 μm. Effective delivery is also dependant on patient education. A patient may be instructed to take a long slow inhalation, which may optionally include a breath hold of 3 seconds or more to minimise exhaled (smaller) particles.

Fine particles, that is, those with an MMAD of less than 10 μm and smaller, tend to be increasingly cohesive and consequently increases the tendency of particles to agglomerate. In the inhaler, agglomeration of fine particles and adherence of such particles to the walls of the inhaler are problems that result in the fine particles leaving the inhaler as large, stable agglomerates, or being unable to leave the inhaler and remaining adhered to the interior of the inhaler, or even clogging or blocking the inhaler.

The uncertainty as to the extent of formation of stable agglomerates of the particles between each actuation of the inhaler, and also between different inhalers and different batches of particles, leads to poor dose reproducibility.

Furthermore, the formation of large stable agglomerates means that the MMAD of the active particles are too large to reach the required part of the lung.

To improve this situation and to provide a desirable consistent FPF and FPD, dry powder formulations often include additive material. The additive material is intended to control the cohesion between particles in the dry powder formulation, and to optimise the de-agglomeration of the active particles back to discrete micronized particles on actuation, on their interaction with the gas jet of the invention as described herein.

Preferably, the additive material is an anti-adherent material and it will tend to reduce the cohesion between particles and will also minimise fine particles becoming attached to the inner surfaces of the inhaler device. The additive materials are often referred to as force control agents (FCAs) and they usually lead to better dose reproducibility and higher fine particle fractions. Known additive materials usually consist of physiologically acceptable material.

Preferred additive materials for used in dry powder formulations include amino acids, peptides and polypeptides having a molecular weight of between 0.25 and 1000 kDa and derivatives thereof. Preferably, the FCA consists substantially of an amino acid, more preferably of leucine, advantageously L-leucine. The D- and DL-forms may also be used. The FCA may comprise or consist of a metal stearate, for example, magnesium stearate. In some embodiments, a plurality of different FCAs can be used.

Dry powder formulations often include coarse carrier particles of excipient material mixed with fine particles of active material. In some formulations, a fine size fraction of a carrier excipient is also included as it is found to enhance drug aerosolization efficiency. In such compositions, rather than sticking to one another, the fine active particles tend to adhere to the surfaces of the coarse and fine carrier particles whilst in the inhaler device, but release and become dispersed and detatched to discrete micronized particles upon actuation of the dispensing device and with interaction of the gas jet, resulting in inhalation into the respiratory tract.

The inclusion of carrier particles is also very attractive where very small doses of active agent are dispensed. It is very difficult to accurately and reproducibly dispense very small quantities of micronized powder and small variations in the amount of powder dispensed will mean large variations in the dose of active agent where the powder comprises mainly active particles. Therefore, the addition of a diluent, in the form of large excipient particles and agglomerates will make dosing more reproducible and accurate.

Carrier particles may comprise or consist of any acceptable excipient material or combination of materials and preferably the material(s) is (are) inert and physiologically acceptable. Advantageously the carrier particles are of a polyol. In particular the carrier particles may be particles of crystalline sugar, for example mannitol, dextrose or lactose. Preferably, the carrier particles are of lactose monohydrate.

Some formulations suitable for use in devices of the invention are already known, for example, those formulations described in WO2001/78696, WO2006/056812, and WO2008053253.

In a third embodiment the device is a soft mist inhaler powered by the propellant energy source.

In a fourth embodiment the device is a nebuliser powered by the propellant energy source.

A device of any of the first to third embodiments may further comprise a Valved Holding Chamber (VHC) or spacer.

The devices of the first to third embodiments may also require one or more metering devices, which one or more metering devices meter the propellant energy source and/or the drug.

The invention is based around the fact that gases can be adsorbed under pressure onto an adsorbant e.g. activated carbon and the greater volumes stored create a power source that can be exploited.

Whilst all aerial gases can be adsorbed, the greater compressibility of carbon dioxide (approx. X10 when adsorbed onto activated carbon at e.g 10 bar) makes it's use particularly attractive.

For example, a canister filled with 25 cm³ of activated carbon and charged with carbon dioxide to reach a pressure of about 10 barg adsorbs about 2.3 g of carbon dioxide (approximately 1.3 litres of gas). Filling the carbon-containing canister with carbon dioxide may be achieved by using either compressed gas (or by adding a weight of solid carbon dioxide calculated to achieve the required pressure). The filled container delivered a total gaseous volume of 1.05 litres of discharge before the pressure of the container reached atmospheric pressure. This compared with only 0.13 litres of delivered gas from the same sized container charged with 10 barg of carbon dioxide, without carbon.

However, depending on the application other aerial gases such as oxygen and nitrogen (approx. ×3 when adsorbed on activated carbon at e.g. 10 bar) may be used, as may e. g carbon dioxide enriched aerial gases. In this regard, by filling a canister with a greater volume of an aerial gas (by adsorbtion) one creates an energy source that can be exploited in novel designs of inhalers and nebulisers as illustrated in the detailed description with reference to a pMDI (FIGS. 7a, 7c and 7d).

The technology is particularly applicable to what in the pharmaceutical industry would be termed a “combination product” namely a device and drug (including a biologic) in combination (typically the FDA approved form).

The term “drug” is defined as a pharmaceutically active component or other medically functional ingredient e.g. saline or a vaccine.

The combination product further comprises a mechanism for releasing a sufficient volume of the propellant at a speed that delivers/aerosolizes a unit dose of drug.

It may also comprises a drug releasing mechanism for releasing a unit dose of formulated drug into a dose metering chamber from which it is delivered.

The key to each device type is the use of the power source, an aerial gas adsorbed on an adsorbent in a canister.

By using such a power source benefits such as:
Compact design;
Ease of use by the patient
Simpler and lower cost design;
Greater consistency of discharge; and
An ability to deliver larger doses than conventionally achieved using the prior art devices
may be achieved.

In accordance with a further aspect of the present invention there is provided a method of delivering a drug to a user via the nasal or pulmonary route comprising administering the drug from a device which uses a propellant energy source to deliver the drug, which propellant energy source is or comprises an aerial gas that is stored in a canister separate of the drug, at a pressure of at least 2 Bar, adsorbed on an adsorbant where it is released therefrom to dispense the drug, which device is absent of a bag-on-valve.

In accordance with yet a further aspect of the present invention there is provided a propellant energy source comprising a 10 to 50 ml canister filed with an aerial gas at a pressure of at least 2 Bar adsorbed on an adsorbent where it is released therefrom to dispense a drug and further comprises a gas delivery mechanism comprising a gas metering chamber with a capacity of 0.2 to 5 ml.

Preferably the delivery mechanism further comprises a metering valve, a valve stem and an actuator.

Preferably it further comprises a gas entry orifice and a gas exit orifice which facilitate the filling and emptying of the gas metering chamber.

As illustrated in, for example FIG. 2 and FIGS. 7a-d, the valve is actuated by the interaction of the cannister and the actuator typically via the valve stem. The gas flow can be controlled by known means, e.g. orifice and conduit size and the use of e.g. reducers, which can alter the flow rate from the chamber.

### BRIEF DESCRIPTION OF THE DRAWINGS

Embodiments of the invention are further described hereinafter with reference to the accompanying drawing, in which:

FIG. 1 is an illustration of a range of prior art inhalers, the drugs typically used therewith, dose range limitations and the form (powder, suspension, solution);

FIG. 2 is an illustrative example of a prior art pressurised metered dose inhaler, its metering valve and adapters for nasal or pulmonary delivery;

FIG. 3 is an illustrative example of a prior art powder inhaler device (passive) with a reservoir delivering multiple doses via a metering mechanism;

FIG. 4 is an illustration of an alternative prior art powder inhaler device (active) with a pump and a mechanism for piercing blisters;

FIG. 5 is an illustration of a prior art soft mist inhaler device and its “uniblock” component;

FIG. 6 is an illustration of a prior art nebuliser;

FIG. 7a is an illustration of an exemplary pressurised metered dose inhaler according to the invention;

FIG. 7b is an illustration of a pMDI, highlighting a standard metering mechanism including a metering valve, meter chamber, actuator valve stem and actuator exit orifice;

FIG. 7c is an illustration of one embodiment of a metering mechanism of the invention showing an enlarged metering chamber in a closed/at rest position;

FIG. 7d is the metering mechanism of FIG. 7c in an open/actuated position;

FIG. 8a-8c are equilibrium adsorbtion isotherms for the gasses oxygen, nitrogen and carbon dioxide on different adsorbents including granular activated carbon (and functionalised varients thereof);

FIG. 9 is a graph illustrating the difference in gas pressure per mass of gas between a canister filed with carbon dioxide alone and one in which the carbon dioxide is adsorded onto activated carbon;

FIG. 10 illustrates the powder dispensing test rig used to test powder delivery;

FIG. 11 illustrates the capsule loading, piercing, and powder dispensing arrangement used in the test rig;

FIG. 12 is a plot of the capsule emptying performance (emitted dose) using a single 200 ms burst of air applied via the test rig; and

FIG. 13 is a photograph showing the effective aerosolization of an exemplary powder for nasal delivery.

### DETAILED DESCRIPTION

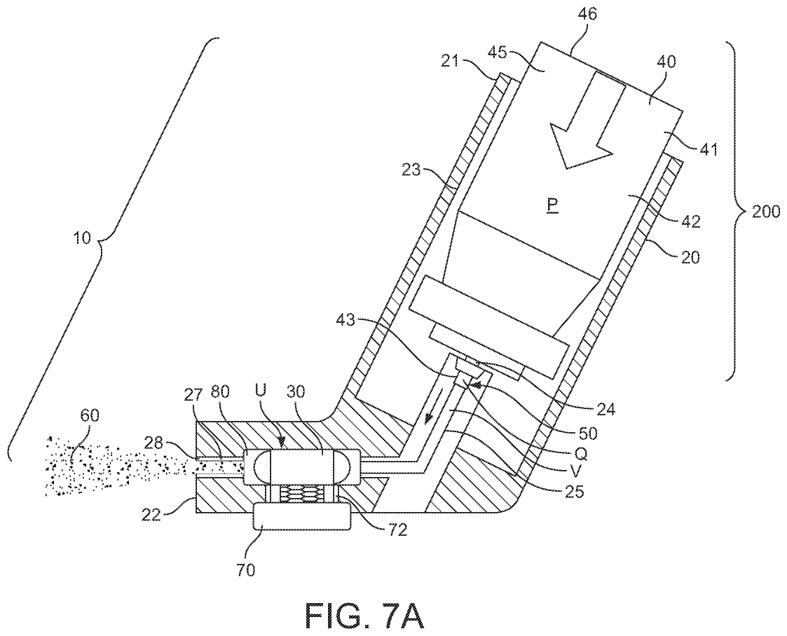

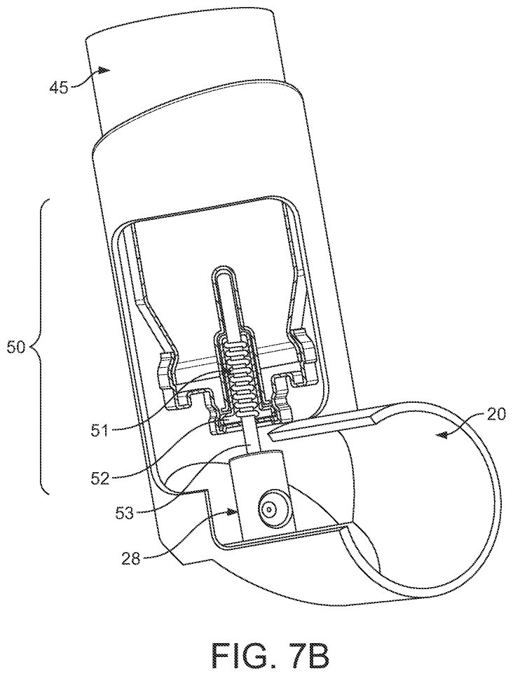

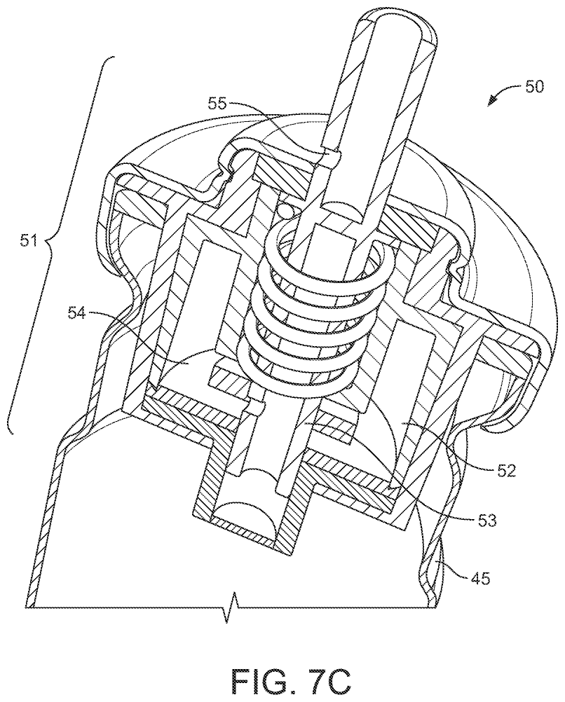

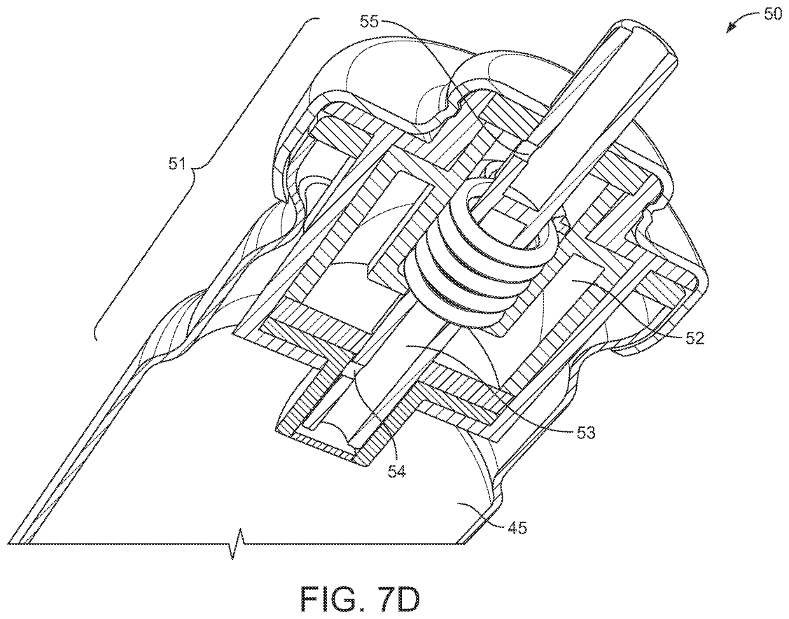

An exemplary combination product (10) or device (20), is illustrated in FIG. 7a.

Whilst the illustration is of a pressurised metered dose inhaler (200) it illustrates the principle of utilising a propellant energy source (40) to deliver the drug (30), which propellant energy source (40) is or comprises an aerial gas (41) that is stored in a canister (45), at a pressure of at least 2 Bar, adsorbed on an adsorbant (42) where it is released therefrom to dispense the drug (30), which device (20) is absent of a bag-on-valve. Where the absorbant (42) is a particulate, a filter or frit (not illustrated) may be employed to ensure any particulates generated by the adsorbant do not leave the canister with the gas stream.

The canister (45) is housed at a proximal (21) end of the device (20) remote from a distal (22) end, which is the end of the device where an aerosol plume (60), on actuation of the device, exits the device (20).

The device (20) comprises canister retaining walls (23) and an end face (24) through which a valve stem (43) of the canister (45) passes. When the canister is actuated, on pressing a base (46) of the canister in the direction of the arrow, gas is released from the canister and can cause a unit dose (U) of the formulated drug (30) to be dispensed.

The gas can be dispensed in a “metered” fashion, with a volume (V) of e.g. from 0.1 to 5 ml, more particularly 0.2 to 2.5 ml, at a pressure (P) of e.g. 2 to 10 Bar which has been demonstrated (Example 1) to effectively aerosolise a high dose (up to 50 mg) of an exemplary powder (Example 2).

In FIG. 7a the gas release mechanism (50) comprises a chamber (52) in operative communication with the canister (45) and device (20) (See FIG. 7b which is applicable to the claimed invention) allowing a known volume of gas, at a given pressure, to be dispensed on actuation of the device (pressing on the canister results in the valve stem being depressed opening the valve—compare e.g. FIGS. 7c and 7d) and the chamber (52) to be refilled with aerial gas (21) as it is desorbed from the adsorbent (42). Thus, a metered dose of aerial gas (41) is released on actuation of a metering valve (51) and is directed from a valve stem (53) along a first conduit (25) to, in the example illustrated, a drug dosing chamber (80) holding a unit (U) dose of a drug (30), which is dispensed in an arosolised plume (60) as the gas travels from upstream of the drug (30) though the dosing chamber (80) where it picks up the drug (in the example illustrated a powder) and carries it along a second conduit (27) to an exit orifice (28) where it leaves the device as a plume (60).

A drug releasing mechanism (70) in this embodiment comprises a piercing mechanism (72) releases the drug (30) from a capsule whereupon it is aerosolised by the gas as it passes from the first conduit (25) through the dosing chamber (80) and out via the second conduit (27) and exit orifice (28).

Whilst FIG. 7a illustrates the delivery of a single unit dose it will be apparent that the drug releasing mechanism (70) could dispense multiple doses using, for example, a carousel, as disclosed in WO2001 17595 (ML Labs) or alternatively Relenza Rotadisc patient instructions available at: https://www.medicines.org.uk/emc/files/pil.3809.pdfDiskhaler or could be a chamber that is refiled from a hopper as the Clickhaler® dry powder inhaler:

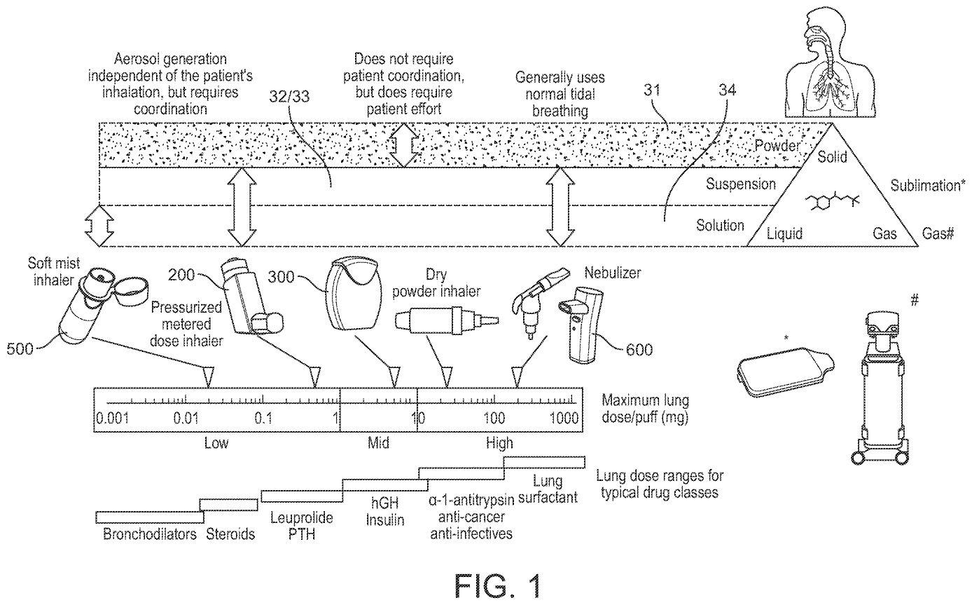

Focussed in vitro proof of principle evaluation of a new chemical entity for asthma”, International Journal of Pharmaceutics 239 (2002) 149-156, illustrated in FIG. 1 therein as a detailed view of the Clickhaler metering cone.

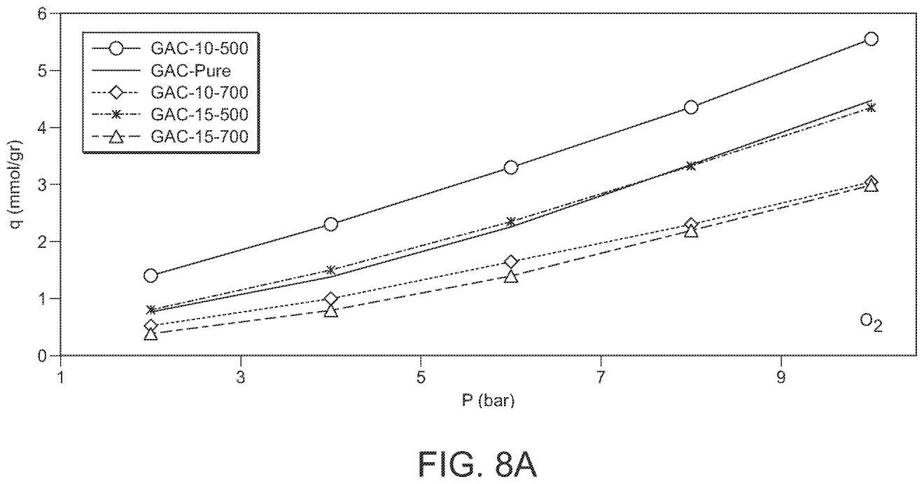

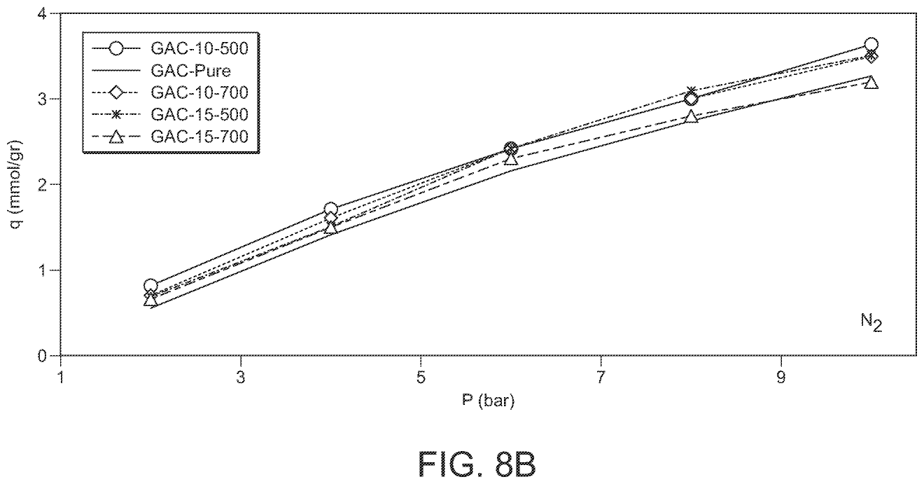

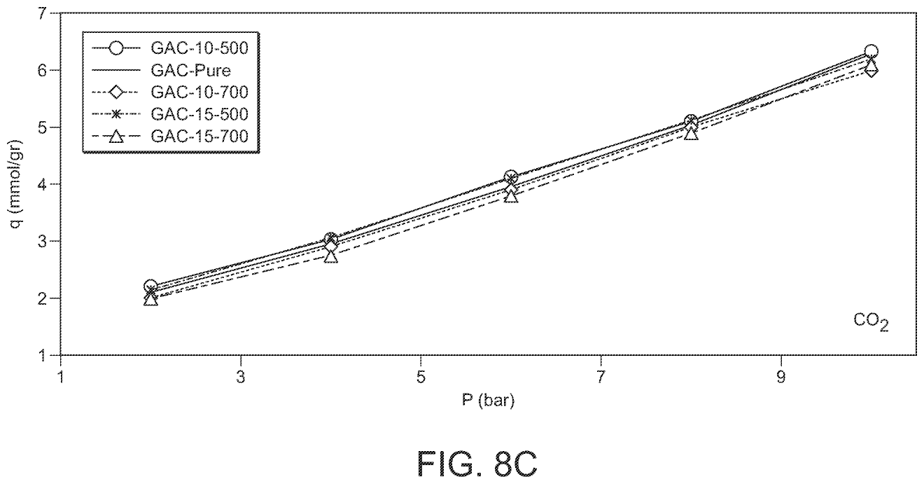

It will also be apparent that the drug to be delivered could also be delivered as a liquid with the dosing chamber (80) being filed with a unit dose of liquid instead of a powder e.g. as per the Respimat® device—Development of Respimat® Soft Mist™ Inhaler and its clinical utility in respiratory disorders, Medical Devices: Evidence and Research 2011:4 145-155, illustrated in FIG. 8 therein.

FIG. 7b illustrates a standard pMDI metering mechanism (50), also applicable to the present invention, and comprises a metering valve (51) fitted to a canister (45) and actuator device (20). It comprises a metering chamber (52) and valve stem (53) leading to an exit orifice (28).

FIGS. 7c and 7d illustrate an exemplary and modified metering mechanism (50) which has a metering chamber (52) with a larger volume capacity than existing pMDIs. It too comprises a metering valve (51) and valve stem (53), the metering valve comprising a gas entry orifice (54) which allows the chamber to be filled from the canister (45) when at rest (FIG. 7c) and a gas exit orifice (55) which allows the gas to exit the chamber (51) when the device (20) is actuated (FIG. 7d).

By way of explanation a typical pMDI canister has a volume of between 10 to 22 ml and the metering mechanism (50) dispenses a liquid and not an aerial gas (21). Because the liquid expands approximately 200 fold on release, in order to deliver a unit dose of the drug (30) which is dissolved or dispersed in the propellant liquid the metering chamber only needs to have a capacity of 20 to 100 μl. In contrast the mechanism (50) employed to deliver, if desired, a metered dose of the pressurised aerial gas is several orders of magnitude larger at 0.1 ml to 5 ml, more typically 0.5 ml to 2.5 ml. This is because the aerial gases expansion is wholly dependent on the pressure at which it is stored.

Thus as 10 bar pressure it will expand 10 fold on release to atmospheric pressure The gas metering mechanism (50), as illustrated in FIGS. 7c and 7d, comprises a metering valve (51), a gas chamber (52), a valve stem (53), a gas entry orifice (54) which is open in an at rest position (7c) and a gas exit orifice (55) which is closed in the rest position.

When actuated, gas entry orifice (54) is closed and gas exits via gas orifice (55) via the valve stem (53).

This principle applies to each of:
A pressurised metered dose inhaler (200);
A dry powder inhaler (300/400);
A soft mist inhaler (500) and
A nebuliser (600) which “generic” devices are all illustrated in FIG. 1 together with information on:
Typical drug type administered with a given device;
Maximum lung dose deliverable under the current limitations of such devices;
Drug form
   Solution (34)
   Suspension (32), dispersions or emulsions
   Powder (31)
User limitations.

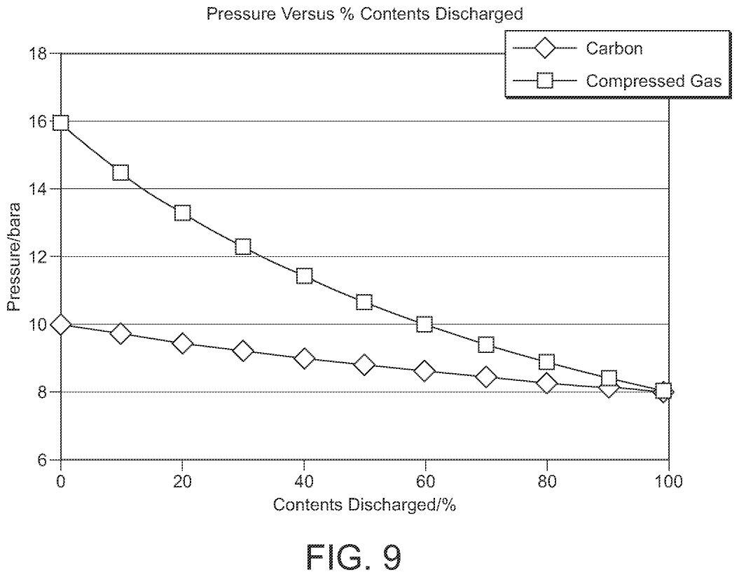

The rational for the invention is further illustrated in FIGS. 8a-8c and FIG. 9.

FIG. 8a-c (taken from ASC Omega 2022, 7, 18409-18428) compares the equilibrium adsorption isotherms of respectively:
Oxygen
Nitrogen; and
Carbon dioxide
   at 25 °C and pressures of from 1 to 10 bar for different adsorbents including granulated activated carbon (GAC)-Pure (and functionalised). What it shows is that significant adsorption can be achieved for each of these gases, with the greatest adsorbtion being for carbon dioxide. It is a fact that one can adsorb and then release significant volumes of these aerial gases from a canister under pressure that allows it to be used as a propellant energy source (40), Such an energy source retains relatively constant pressure over the life of the canister as illustrated in FIG. 9. The different gases can be used alone or in combination.

FIG. 9, from WO2008064293, compares the content discharge from a canister of carbon dioxide when adsorbed v as a compressed gas. What it demonstrates is that an adsorbed gas makes it easier to manage “controlled” dosing.

In one embodiment as shown in FIGS. 7a to 7d, the canister may be of the same dimensions as for a prior art (10 to 50 ml, typically 10 to 22 ml). Preferably it is filled to at least 60%, through 70%, 80% to 90% of more of its volume.

Thus, an exemplary drug for use in a modified pMDI inhaler of the invention is Salbutamol. Salbutamol is a drug used for asthma treatment. Thus, a prior art device may contain the drug and propellant in a canister with an internal volume of 20 ml, which is sufficient to administer up to 100-200 metered doses of medication.

Surprisingly, the Applicants have determined that a similarly-dimensioned canister using an activated carbon absorbant, and compressed to ~2 to 10 bar with CO₂ as the propellant gas, may also be used to dispense a very significant number of doses of powder from pierced capsules. Thus, the dimensions, familiar shape, and portability of existing asthma inhalers may be potentially carried over to a similar design, but using a zero GWP propellant.

Note that although a standard metering valve is shown in the embodiment in FIG. 7b, a metering valve as such may not be required with the Applicant's invention. For this embodiment all that is required is to pass a sufficient minimum volume of gas to dispense the e.g. salbutamol from e.g. a pierced capsule to dispense a repeatable dose of the drug, and such minimum gas volume may not be require to be precisely metered. The minimum gas volume varies depending on the mass of powder in the exemplary capsule. For capsules containing from 10 to 50 mg of powder, the Applicant has found such minimum gas volume may be in the range of 1-10 ml of gas (at atmospheric pressure).

Furthermore, the Applicants have determined that a number of alternative aerial gases may be used as illustrated by the data in FIG. 8.

Furthermore, as noted from FIG. 9 where a canister contains a compressed gas only (black rectangles), the canister must be compressed to a very high initial pressure of 16 bars in order to have a sufficient final working pressure of 8 bars at the end of its life. This pressure is too high for an existing low-cost aluminium deep-drawn canister used in an inhaler. As a result a much more expensive and bulky thick-walled CO₂ cartridge would need to be used to allow 16 bars to be stored. Such cartridges are too expensive and heavy for a portable and disposable inhaler. However in the case of an activated carbon canister (black diamonds), an initial pressure of up to 10 bars could be used, which is suitable for a low-cost aluminium deep drawn canister.

Additionally, as noted from FIG. 9, for the compressed gas only case, the pressure drops in half from the beginning to the end of the canister life. This would cause a substantial variation in entrainment air flow and therefore affect dose repeatability over the life of the canister. This problem is resolved by the activated carbon canister where the pressure drop is only 20% over the working life of the canister.

Consequently, the embodiment shown in FIGS. 7a to 7d solves many problems associated with each of the different various prior art inhalers, particularly in relation to the pressing problem of high carbon footprint.

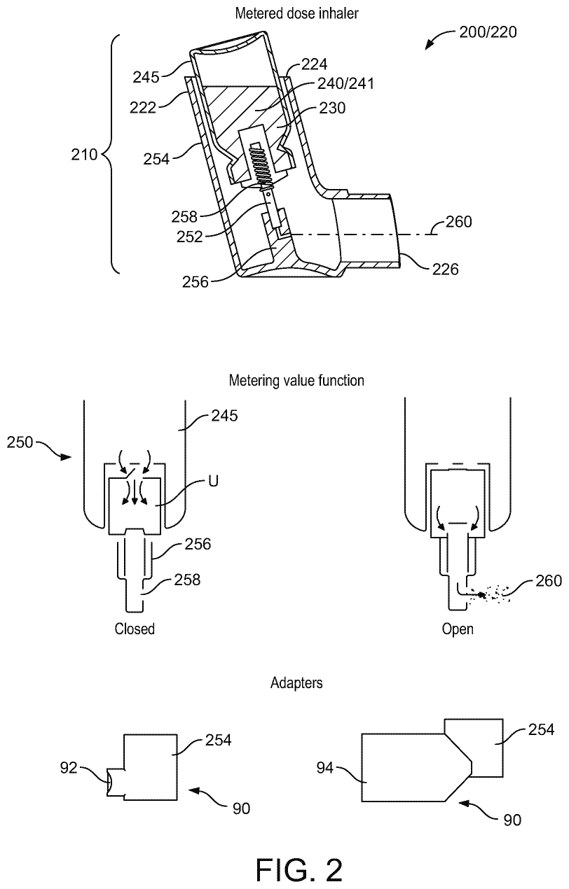

Each of FIGS. 2 to 6 are included to provide context to how the invention illustrated with reference to FIGS. 7a to 7d (and further supported by FIGS. 8 and 9) may be applied to modify these alternative prior art devices many of the features of which may remain unchanged.

Thus, refering to FIG. 2, a prior art pressurised metered dose inhaler (200) is a combination product (210) comprising a device (220), a drug (230) and a power source (240) or propellant (241). The drug (230) and propellant (241) a liquid, typically a hydrofluroalkane (HFA), are retained together in a sealed canister (245) under pressure (P). The device (220) has an actuator body (222), a first opening (224) that receives the canister (245), and a second opening (226) out of which the aerosolised (260) drug (230) exits the device (220). The device (220) has a metering mechanism (250) for delivering a metered dose comprising a metering valve (252), actuator (254), actuator seat (256) and actuator nozzle (258).

Actuation of the device results in the delivery of a metered dose, the liquid propellant expanding and aerosolising the drug which is dispensed through an appropriate adapter (90) (nasal (92) or mouthpiece (94)).

In contrast to such a prior art device, and as illustrated in FIG. 7a to 7d, instead of a liquid propellant the device is powered by the release of an aerial gas from an adsorbent from a canister which delivers a unit dose of drug which is housed separate of the propellant.

By controlling the volume (V), and flow rate (Q) of release of the gas a unit dose of a drug can be delivered.

The drug can take the form of a powder, suspension, dispersion or emulsion, or solution.

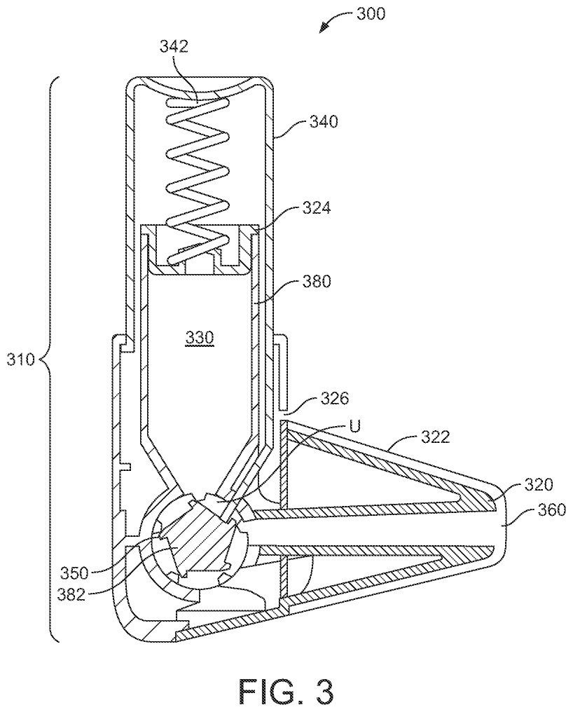

Similarly, refering to FIG. 3, a prior art dry powder inhaler (300) is a combination product (310) comprising a device (320), a drug (330) and a power source (340) such as a mechanical spring (342). The drug (330) is stored in a bulk chamber (380) which is delivered to a metering chamber (382). The device (320) has a body (322). An overcap (324) at one end houses the power source (340) and the drug (330) is fed from the bulk chamber (380) into a metering chamber (382) of a drug metering mechanism (350). Channels (326) in the device ensure that when a user inhales, air is drawn across the device and aerosolised (360) drug (330) exits the device (320).

Such a device can be modified by way of the invention such that a unit dose of a powdered drug (330) is released from a chamber actively (as opposed to passively) using an aerial gas dispensed from a power source (40) as illustrated in FIGS. 7a to 7d.

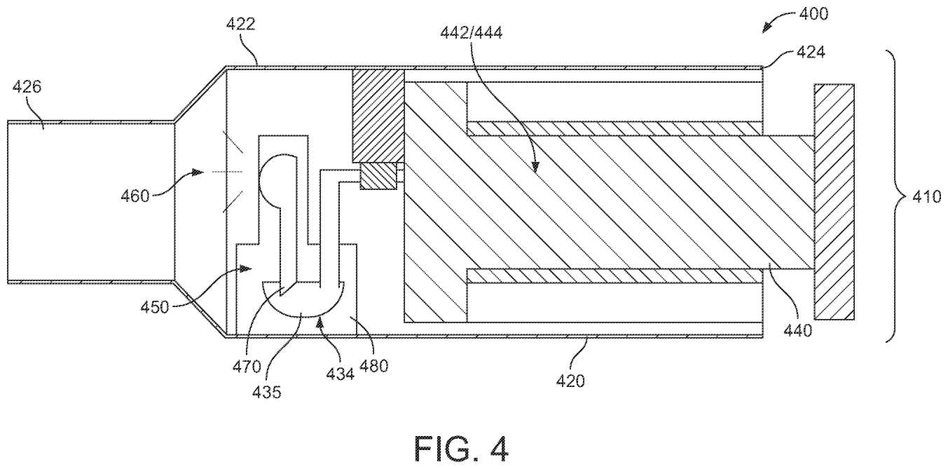

Similarly, refering to FIG. 4, a prior art active dry powder inhaler (400) is a combination product (410) comprising a device (420), a drug (435) and a power source (440) in the form of a pump (442) and integral air reservoir (444). The drug (435) is stored in a chamber (480) in a unit dose form such as a blister (434) and is released from the chamber by a blister piercing mechanism (470). The device (420) has a body (422), a first opening (424) which receives the pump (442), and a second opening (426) out of which the aerosolised (460) drug (435) exits the device (420).

Such a device can be modified by way of the invention such that a unit dose of a powdered drug (435) is released from a chamber actively (as opposed to passively) using an aerial gas dispensed from a power source (440) as exemplified in FIGS. 7a to 7d which replaces the pump and integral air resevoir.

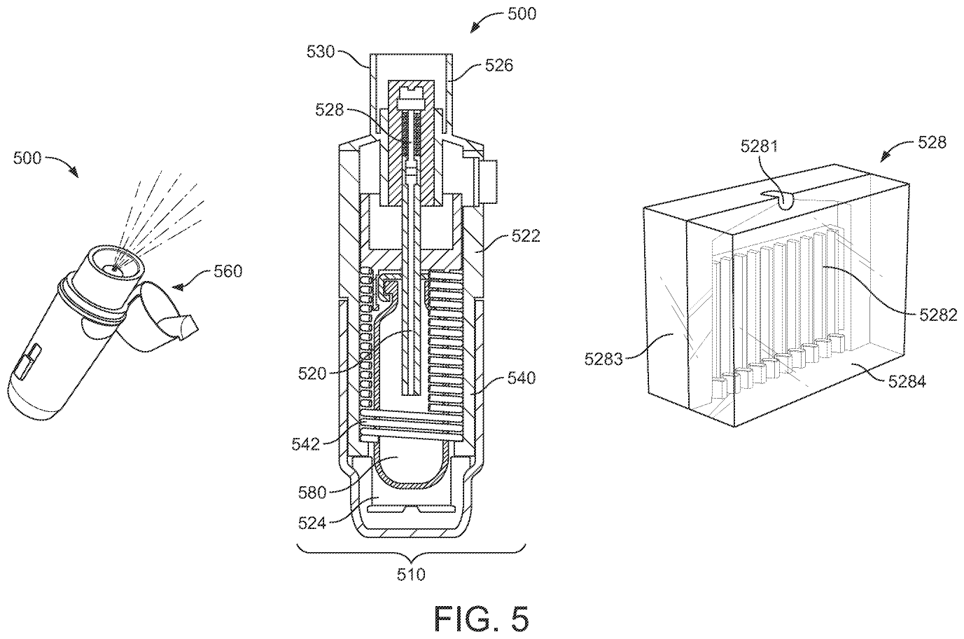

Similarly, refering to FIG. 5, a prior art active soft mist inhaler (500) is a combination product (510) comprising a device (520), a drug (530) and a power source (540) in the form of a spring (542) which drives a unit dose of drug (530) from a chamber (580) through a uniblock (528) where it is aerosolised (560).

The device (520) has a body (522), a first end (524) which houses the spring (542) and a second opening (526) out of which the aerosolised (560) drug (530) exits the device (520), the dose being held in a chamber (580) intermediate the two with the uniblock (528) positioned beyond the dosing chamber.

The uniblock (528) comprises a nozzle outlet (5281), filter structure (5282), silicon wafer (5283) and glass (5284).

Such a device can be modified by way of the invention such that a unit dose of a powdered drug (530) is released from its chamber actively using an aerial gas dispensed from a power source (40) as exemplified in FIGS. 7a to 7d which replaces the spring (542).

It may be further possible to replace the monolithically-fabricated uniblock with a cheaper nozzle alternative as a longer dose delivery time (t) (comparable to this method of delivery) may be achieved by controlling the timed release of the aerial gas from the dispenser. This may also result in less pressure being required through the nozzle to generate the soft mist.

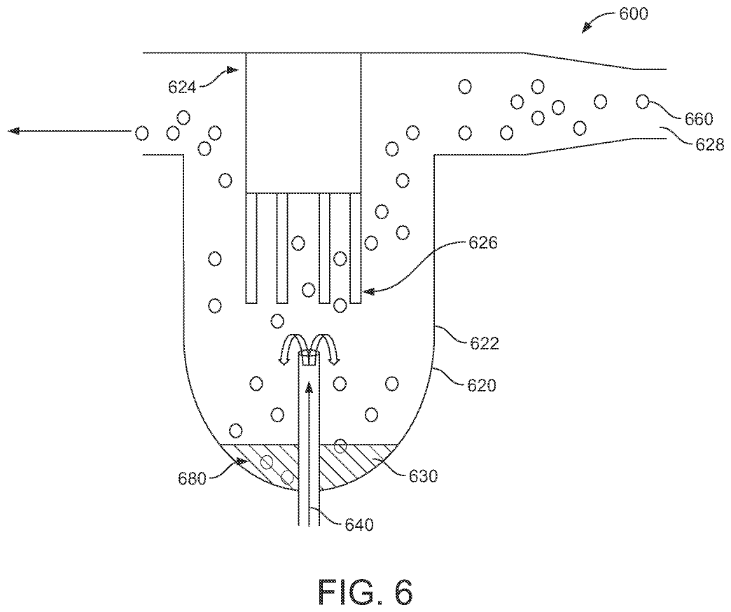

Finally, referring to FIG. 6 a representative prior art neubuliser (600) comprises a device (620), a drug (630) and a power source (640) pressurised gas from a compressor (electric). The drug (630) is stored in a resevoir (680) and is aerosolised. The device (620) has a body (622). The device (620) has a body (622), an air inlet (624) and an air outlet (628) or mouthpiece out of which the aerosolised (660) drug (630) exits the device (620). The device further comprises a baffle arrangement (626) to assist aerosolization and break up.

In use the pressurised gas (air) aerosolises the liquid medication in the reservoir and as ambient air is drawn across the device to the mouthpiece it entrains aerosolided particles which are delivered to the user. Some drug loss occurs through the inlet.

Such a device can be modified by way of the invention such that the drug (630) is caused to arosolise using an aerial gas dispensed from a power source (40) as exemplified in FIGS. 7a to 7d as opposed to air driven electrically through a compressor. Thus, it does not require mains electric or batteries for operation.

### EXAMPLE 1

### Powder Formulation

A powder particle containing Fluorescein formulation suitable for nasal delivery was prepared based on the methodology of Suhaidi et al (2023)—Bulk Flow Optimisation of Amorphous Solid Dispersion Excipient Powders through Surface Modification. Pharmaceutics, 15.

The particles when dissolved in artificial mucus would fluoresce brightly under UV light in a clear nasal cast. As the particles were for nasal delivery a particle size distribution with the largest proportion of the particles in the range 10 μm to 40 μm was prepared.

A blend of 90% Maltodextrin, 5% L-leucine, and 5% Fluorescein produced a powder with particle diameter in the correct size range for nasal delivery, and with good flowability. The particles formulated became a bright green colour under UV.

The size distribution of the resulting optimised fluorescent powder blend was measured on a Microtrac Camsizer X2 particle size and shape analyser, with three repeats. The size range was Dv10=15.1 μm; Dv50=23.7 μm; Dv90=35.8 μm. These particle sizes measured by the Camsizer are geometric median diameters. These may be considered to be approximately the same as the aerodynamic diameter as particles were spherical and had bulk density of approximately unity.

### EXAMPLE 2

### Aerosolisation of the Powder

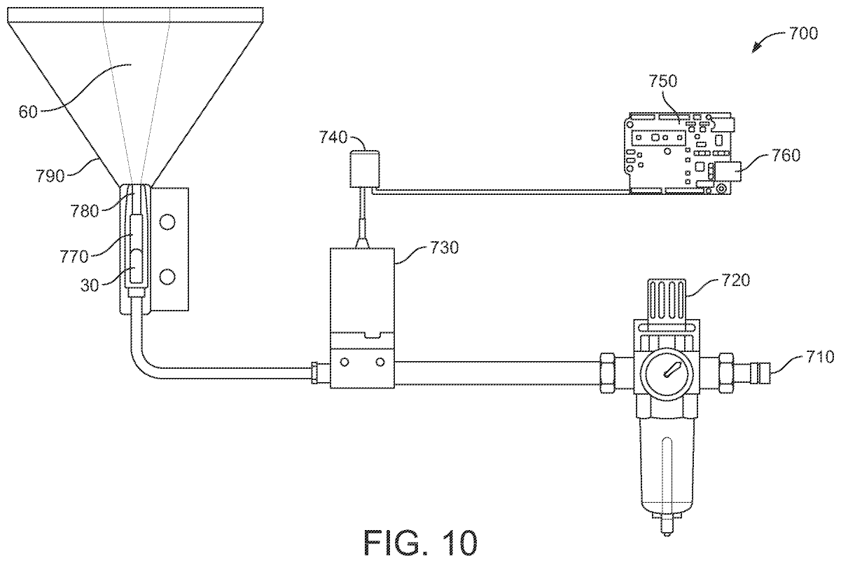

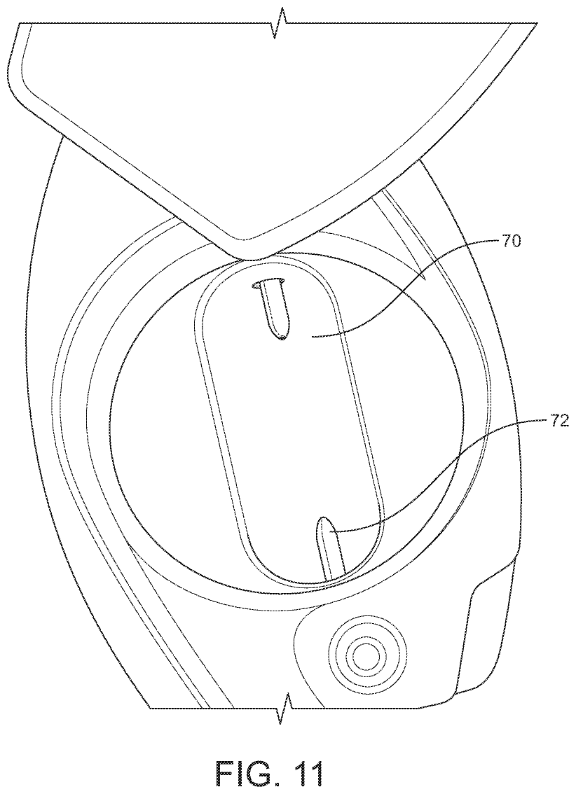

To demonstrate that the power source can aerosolise particles, whether for nasal or pulmonary delivery, the larger, heavier nasal particles (size range 10 to 40 μm), as opposed to lighter, smaller pulmonary particles (size range 2 to 10 μm) were used, placed in capsules and inserted into a powder dispensing test rig as illustrated in FIG. 10 and described further below. The rig (700) comprises an air inlet (710), an adjustable regulator (720) and a solenoid valve (730) which is connected to an Arduino controller (750) linked to a computer (760) via a relay (740). A pierced capsule (30) is placed in a chamber (770) see also FIG. 11 and a controlled pulse of air is released such that the drug (30) is delivered as a plume (60) out of an insufflator nozzle (780) onto a plate inside a conical funnel (790) where it can be visualised under UV.

### Nasal Powder Dispensing Device

Dry powder inhalers (DPIs) for pulmonary inhalation therapy are devices somewhat similar to nasal powder delivery devices as they both release dry powder particles from a reservoir into an aerosol plume for delivery to the patient. However, particle size properties are different for pulmonary inhalation, with particles in the range 2 μm-5 μm being regarded as optimal for topical respiratory delivery (Islam, N., & Gladki, E. (2008). Dry powder inhalers (dpis)—a review of device reliability and innovation. International Journal of Pharmaceutics, 360, 1-11).

It is known that DPIs may release drugs in powder from either
i) capsules;
ii) multi-dose strips, or
iii) bulk powder reservoirs.

There are several capsule-based DPIs on the market and they have some advantages over powder reservoir devices as the capsule and blister packaging protects the powder contents from moisture. Furthermore, capsule filling lines are widely used and deployed in the pharmaceutical industry.

Capsule-based DPIs also have good dose-to-dose consistency (Islam & Gladki, 2008). Well-known capsule-based DPIs which have been on the market for many years include the Breezhaler® (Novartis, Basel, Switzerland); the HandiHaler® (Boeringer Ingelheim, Germany); the Twister® (Aptar, IL, USA); and the Rotahaler® (Cipla, Mumbai, India). Consequently, Applicant chose a nasal powder delivery device which used Fluorescein dyed particles loaded into capsules for testing.

The Plastiape RS01 is another example of a commercial capsule-based DPI (hereon called cDPIs). Like almost all DPIs, it is a passive device that requires users to activate the device by oral inspiration to allow inhalation of the released drug particles (Elkins, M. R., Anderson, S. D., Perry, C. P., Daviskas, E., & Charlton, B. (2014) Inspiratory flows and volumes in subjects with non-cf bronchiectasis using a new dry powder inhaler device. Open Respir Med J, 8, 8-13). The capsule loading, piercing, and powder dispensing arrangement (70) comprises two hollow stainless steel piercing spikes (72) which penetrate each of the opposite domed ends of a size 3 gelatin capsule (FIG. 11). The piercing spikes create holes in the capsule of diameter 1.15 mm (Martinelli, F., Balducci, A. G., Rossi, A., Sonvico, F., Colombo, P., & Buttini, F. (2015). “pierce and inhale” design in capsule based dry powder inhalers:

Effect of capsule piercing and motion on aerodynamic performance of drugs. International Journal of Pharmaceutics, 487, 197-204). Users deploy these hollow spikes by depressing two opposed spring-loaded buttons. Whilst the passive method of oral inhalation to induce powder delivery does not suit a nasal delivery device the RS01 device does have a capsule piercing arrangement which is be suitable for capsule emptying. A similar method of piercing capsules is described in (Farkas, D., Hindle, M., & Longest, P. W. (2017). Development of an inline dry powder inhaler that requires low air volume. Journal of Aerosol Medicine and Pulmonary Drug Delivery, 31, 255-265). In this latter work, the authors described using a “straight through” airflow design where a pulse of air pushes the powder contents out of the capsule in a longitudinal direction.

### Measuring the Emitted Dose From the Capsules

Because of the simplicity of the approach described by (Farkas et al., 2017), Applicant used the “straight through” capsule piercing approach in a custom nasal powder delivery device. A manual “squeeze bulb” powder insufflator was procured (Sheehy House Insufflator, Grace Medical, Memphis USA). The manual “squeeze bulb” was replaced by a programmable air pulse source as part of a test rig described earlier with reference to FIG. 10. To test capsule emptying performance, capsules were filled with 10, 20, 30, 40 and 50 mg of the fluorescent powder blend. Five repeated tests were made at each of the 10 mg mass increments. Hence a total of 25 capsules were tested. Each capsule had its domed ends pierced using an RS01 device shown in FIG. 11 having a dosing chamber (70) with a piercing mechanism (72) in which a capsule is placed. Each empty capsule was firstly weighed on an analytical balance before filling to determine a mass m1. Capsules were secondly weighed after filling to determine a mass m2. The mass of powder in the capsule mp prior to dispensing was then calculated as (m2−m1). After dispensing of a single 200 ms burst of air through the capsule at 1 barg pressure from the test rig, the emptied capsule was weighed again on the analytical balance to determine a mass m3. The mass of powder dispensed from the capsule md was then calculated as follows:

md=mp−(m3−m1)

Finally, the emitted dose (ED) as a percentage was calculated as:

ED=100·(md/mp)

### Results

### Capsule Emptying Performance

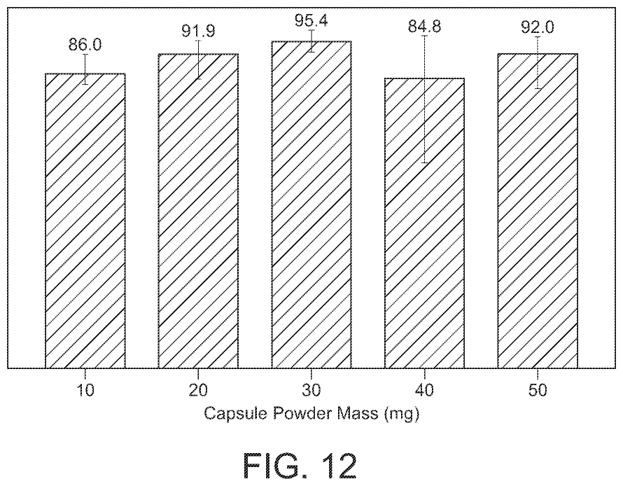

FIG. 12 shows a plot of the capsule emptying performance (emitted dose) using a single 200 ms burst of air applied via the test rig. This chart demonstrates effective capsule emptying performance for each mass increment. Only one relatively poor emptying result (60%) for 1 of the 25 capsules tested (40 mg powder mass) measured, which caused the lower percentage.

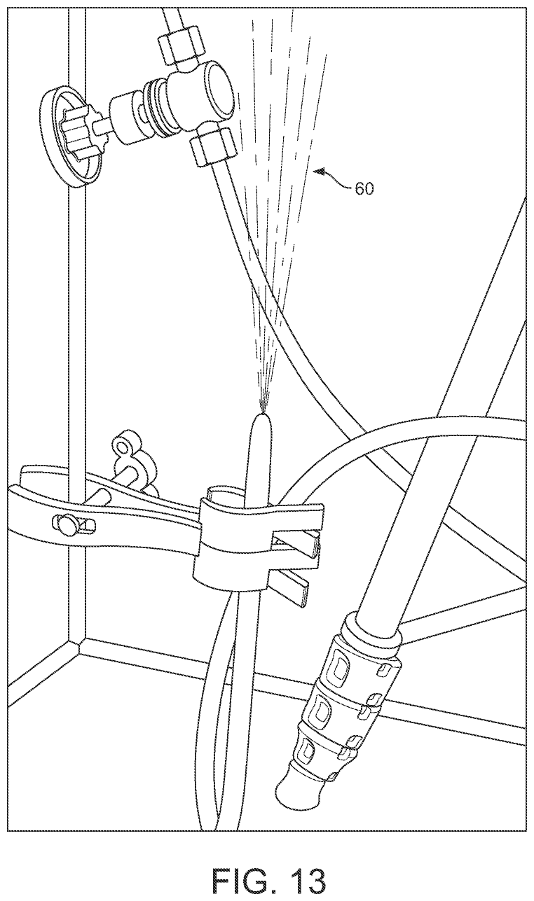

A photograph of the plume (60) emitted is illustrated in FIG. 13.

1. A device for delivering a drug to a user via a nasal or pulmonary route, wherein the device comprises: a propellant energy source to deliver the drug, the propellant energy source is or comprises an aerial gas that is stored in a canister separate of the drug, at a pressure of at least 2 Bar, adsorbed on an adsorbant where it is released therefrom to dispense the drug, wherein the device is absent of a bag-on-valve in the canister.

2. A device as claimed in claim 1, wherein the aerial gas is air, oxygen, nitrogen, or carbon dioxide.

3. A device as claimed in claim 2, wherein the aerial gas is carbon dioxide or is enriched with carbon dioxide.

4. A device as claimed in claim 1, wherein the propellant energy source is positioned in the device upstream of the drug, such that on actuation of the canister a unit dose of the drug is aerosolized from the device in a controlled manner.

5. A device as claimed in claim 4, further comprising a gas metering mechanism for releasing a sufficient volume of the aerial gas at a flow rate that delivers and aerosolizes the unit dose of the drug.

6. A device as claimed in claim 5, wherein the gas metering mechanism comprises a metering valve, a metering chamber, a valve stem and an actuator.

7. A device as claimed in claim 6, wherein the gas metering mechanism further comprises a gas entry orifice and a gas exit orifice thereby allowing the metering chamber to be filled from the canister and emptied to the device.

8. A device as claimed in claim 1, further comprising a drug release mechanism for releasing a unit dose of the drug from a unit dose drug chamber from which the unit dose is delivered to a subject.

9. A device as claimed in claim 8, wherein the drug release mechanism comprises a capsule piercing mechanism.

10. A device as claimed in claim 8, wherein the drug release mechanism comprises a carousel to release the unit dose of the drug from a bulk drug chamber to the unit dose drug chamber.

11. A combination product comprising the device as claimed in claim 1 and a drug, wherein the drug, including a biologic, is a regulatory approved medicine and is present in the device as either:
a) a dry powder;
b) a multi-phase dispersion of a solid in a liquid or a liquid in a liquid; or
c) a single-phase solution.

12. A combination product as claimed in claim 11, wherein the device is a pressurised metered dose inhaler powered by the propellant energy source.

13. A combination product as claimed in claim 11 wherein the drug is the dry powder and
the device is an active dry powder inhaler, powered by the propellant energy source.

14. A combination product as claimed in claim 11, wherein the drug is the multi-phase dispersion of the solid in the liquid or the liquid in the liquid, or the single-phase solution, and the device is a soft mist inhaler powered by the propellant energy source.

15. A combination product as claimed in claim 11, further comprising a mouthpiece adapter or nasal adapter.

16. A combination product as claimed in claim 12, wherein the drug is provided in a bulk chamber.

17. A combination product as claimed in claim 12, wherein the device comprises a drug release mechanism for releasing a unit dose of the drug from a unit dose drug chamber from which the unit dose is delivered to a subject, wherein the drug is provided in a sealed unit dosage form and the drug release mechanism is a piercing mechanism.

18. A combination product as claimed in claim 17, wherein the sealed unit dosage form comprises a blister pack or a capsule.

19. A combination product as claimed in claim 11, wherein the device is a nebuliser powered by the propellant energy source.

20. A combination product as claimed in claim 11, further comprising a frit or filter in a gas discharge portion of the canister.

21. A combination product as claimed in claim 11, further comprising a Valved Holding Chamber (VHC) or spacer.

22. A combination product as claimed in claim 11, wherein the drug is selected from one or more of the group consisting of:
i. long acting muscarinic antagonists (LAMA),
ii. long acting beta agonists (LABA),
iii. short acting beta-2 agonists (SABA); and
iv. corticosteroids.

23. A method of delivering a drug to a user via the nasal or pulmonary route, comprising: administering the drug from a device which uses a propellant energy source to deliver the drug, the propellant energy source is or comprises an aerial gas that is stored in a canister separate of the drug, at a pressure of at least 2 Bar, adsorbed on an adsorbant where it is released therefrom to dispense the drug, wherein the device is absent of a bag-on-valve in the canister.

24. A propellant energy source, comprising: a 10 to 50 ml canister filed with an aerial gas at a pressure of at least 2 Bar adsorbed on an adsorbent where it is released therefrom to dispense a drug, wherein the cannister comprises a gas delivery mechanism comprising a metering chamber with a capacity of 0.2 to 5ml.

25. A propellant energy source as claimed in claim 24, wherein the gas delivery mechanism further comprises a metering valve and valve stem.

26. A propellant energy source as claimed in claim 25, wherein the gas delivery mechanism further comprises a gas entry orifice and a gas exit orifice thereby allowing the metering chamber to be filled from the canister and delivered to a device.
